(19) United States
(12) Patent Application Publication
(10) Pub. No.: US 2020/0361845 A1
(43) Pub. Date: Nov. 19, 2020

(54) Preparation and Use of Biphenyldicarboxylic Acids

(71) Applicant: ExxonMobil Chemical Patents Inc., Baytown, TX (US)

(72) Inventors: Etienne Mazoyer, Woluwe Saint Pierre (BE); Jarid M. Metz, Doylestown, PA (US); Michael P. Lanci, Flemington, NJ (US)

(21) Appl. No.: 16/960,617

(22) PCT Filed: Oct. 30, 2018
(86) PCT No.: PCT/US2018/058161

§ 371 Date: Jul. 8, 2020

Related U.S. Application Data
(60) Provisional application No. 62/625,107, filed on Feb. 1, 2018.

(51) Int. Cl. C07C 51/265; B01J 27/128

(52) U.S. Cl. CPC C07C 51/265; C07C 63/333; B01J 27/128

(57) ABSTRACT

A process for selective oxidation of at least one dimethylbiphenyl compound to the corresponding biphenyldicarboxylic acid, where the dimethylbiphenyl compound is supplied to at least one reaction zone together with an acidic solvent, an oxidizing medium, and a catalyst comprising cobalt, manganese, and bromine. The dimethyl biphenyl compound and oxidizing medium are contacted with the catalyst in the at least one reaction zone at a temperature of 150 to 210° C. to oxidize the dimethylbiphenyl compound to the corresponding biphenyldicarboxylic acid. The supply of dimethylbiphenyl compound to the at least one reaction zone is then terminated, but the supply of oxidizing medium and catalyst is continued with the at least one reaction zone at a temperature of 150 to 210° C. A reaction product comprising at least 95 wt % of the biphenyldicarboxylic acid based on the total weight of oxidized dimethylbiphenyl compound is then recovered from the at least one reaction zone.

### PRIORITY

This application claims the benefit of Provisional Application No. 62/625,107, filed Feb. 1, 2018, the disclosure of which is incorporated herein by reference.

### FIELD

This disclosure relates to the preparation and use of biphenyldicarboxylic acids.

### BACKGROUND

Biphenyldicarboxylic acids (BPDAs), especially the 3,4′ and 4,4′ isomers, are useful intermediates in the production of a variety of commercially valuable products, including polyesters and plasticizers for PVC and other polymer compositions. For example, BPDAs can be converted to ester plasticizers by esterification with a long chain alcohol. In addition, biphenyldicarboxylic acids are potential precursors, either alone or as a modifier for polyethylene terephthalate (PET), in the production of polyester fibers, engineering plastics, liquid crystal polymers for electronic and mechanical devices, and films with high heat resistance and strength.

As disclosed in U.S. Pat. Nos. 9,580,572 and 9,663,417, the entire disclosures of which are incorporated herein by reference in their entirety, BPDAs may be produced by hydroalkylation of toluene followed by dehydrogenation of the resulting (methylcyclohexyl)toluene (MCHT) to produce dimethylbiphenyl (DMBP) compounds. The resultant DMBP compounds can then be oxidized to the desired diacids by any known method, for example reaction with an oxidant, such as oxygen, ozone or air, or any other oxygen source, such as hydrogen peroxide, in the presence of a catalyst, such as Co and/or Mn, at temperatures from 30° C. to 300° C.

Alternative routes via benzene are described in U.S. Pat. No. 9,085,669, in which the benzene is initially converted to biphenyl, either by oxidative coupling or by hydroalkylation to cyclohexyl benzene (CHB) followed by dehydrogenation of the CHB, and then the biphenyl is alkylated with methanol. The resultant DMBP compounds can then be oxidized to the desired diacids by the method described above.

Although significant research has recently been conducted on the production and purification of DMBP compounds, especially in relation to enhancing the yield of the 3,4′- and especially the 4,4′-isomer, little research has been focused on the subsequent oxidation step to produce the diacid. Instead, current proposals rely on the commercially available processes for oxidizing alkylbenzenes, such as toluene, xylene and pseudocumene, which have been available for over 50 years. For example, U.S. Pat. No. 2,833,816 discloses the liquid-phase oxidation of p-xylene to terephthalic acid in the presence of a cobalt/manganese/bromine complex catalyst.

More recently, U.S. Pat. No. 6,476,257 has proposed producing aromatic carboxylic acid from alkylaromatics by oxidation in acetic acid as solvent with an oxygen-containing gas in the presence of cobalt/manganese/bromine complex catalyst, wherein nickel and carbon dioxide are added to increase the activity of the cobalt/manganese/bromine complex catalyst.

The process was conducted either on a batch or continuous basis. Whereas a long list of different alkylaromatic compounds, including 4,4′-dimethylbiphenyl, are disclosed, all the Examples involve oxidation of p-xylene to terephthalic acid.

There is, therefore, interest in developing oxidation processes which are optimized for the conversion of dimethylbiphenyl compounds to the corresponding biphenyldicarboxylic acids and where the co-production of under-oxidized species, such as the monocarboxylic acid and the aldehyde acid, is minimized.

### SUMMARY

The present disclosure provides a process for selective oxidation of at least one dimethylbiphenyl compound to the corresponding biphenyldicarboxylic acid, the process comprising:

(a1) supplying at least one dimethylbiphenyl compound, an acidic solvent, an oxidizing, medium, and a catalyst comprising cobalt, manganese, and bromine to at least one reaction zone;

(b1) contacting the at least one dimethylbiphenyl compound and the oxidizing medium with the catalyst in the at least one reaction zone at a temperature of 150 to 210° C. to oxidize the at least one dimethylbiphenyl compound;

(c1) ceasing the supply of the at least one dimethylbiphenyl compound but continuing the supply of the oxidizing medium and catalyst to the at least one reaction zone at a temperature of 150 to 210° C.; and

(d1) recovering from the at least one reaction zone a reaction product comprising at least 95 wt % of the corresponding biphenyldicarboxylic acid based on the total weight of oxidized dimethylbiphenyl compound.

Advantageously, a polyester product may be produced from the reaction of a diol with at least one of the following;
(i) the biphenyldicarboxylic acid recovered in (d1) and/or
(ii) a biphenyldiester product formed from the esterification of the biphenyldicarboxylic acid recovered in (d1).

The present disclosure also provides a process for selective oxidation of 3,4′- and/or 4,4′-dimethylbiphenyl to 3,4′- and/or 4,4′-biphenyldicarboxylic acid, the process comprising:

(a2) supplying 3,4′- and/or 4,4′-dimethylbiphenyl, an acidic solvent, an oxidizing medium, and a catalyst comprising cobalt, manganese, and bromine to at least one reaction zone;

(b2) contacting the 3.4′- and/or 4,4′-dimethylbiphenyl and the oxidizing medium with the catalyst in the at least one reaction zone at a temperature of 150 to 210° C. to oxidize the 3,4′- and/or 4,4′-dimethylbiphenyl

(c2) ceasing the supply of the 3,4′- and/or 4,4′-dimethylbiphenyl but continuing the supply of the oxidizing medium and catalyst to the at least one reaction zone at a temperature of 150 to 210° C.; and

(d2) recovering from the at least one reaction zone a reaction product comprising at least 95 wt % of 3,4′- and/or 4,4′-biphenyldicarboxylic acid based on the total weight of oxidized 3,4′- and/or 4,4′-dimethylbiphenyl.

### DETAILED DESCRIPTION

As used herein, “wt %” means percentage by weight, “vol %” means percentage by volume, “mol %” means percentage by mole, “ppm” means parts per million, and “ppm wt” and “wppm” are used interchangeably to mean parts per million on a weight basis. All “ppm” as used herein are ppm by weight unless specified otherwise. All concentrations herein are expressed on the basis of the total amount of the composition in question. Thus, the concentrations of the various components of the first mixture are expressed based on the total weight of the first mixture. All ranges expressed herein should include both end points as two specific embodiments unless specified or indicated to the contrary.

As used herein, the term dimethylbiphenyl (DMBP) refers to compounds having the general chemical structure:

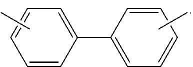

For convenience, the structures below are shown as the 4,4′-isomers, but it will be understood that the 3,3′-, 4,3′- and 3,4′-isomers, 2,2′-isomers, 2,3′- and 3,2′-isomers, and 2,4′- and 4,2′-isomers of these compounds are also covered by the general terminologies.

The term “M-Acid” refers to a mono-carboxylic acid of a DMBP molecule. The chemical structure of methyl-1,1′-biphenyl-carboxylic acid is:

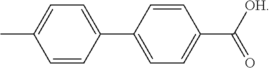 [SMILES: Cc1ccc(-c2ccc(C(=O)O)cc2)cc1]

The term “M-Ald” refers to a mono-aldehyde of a DMBP molecule, which has the following chemical structure:

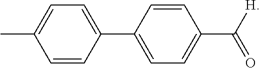

The term “Ald-Acid” refers to a biphenyl molecule having an aldehyde substituent on one ring and an acid substituent on the other ring, which has the following chemical structure:

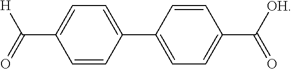 [SMILES: O=Cc1ccc(-c2ccc(C(=O)O)cc2)cc1]

The terms “Diacid” refers to a biphenyl molecule having carboxylic acid substituents on each ring, which has the following chemical structure.

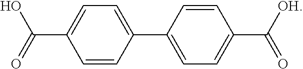 [SMILES: O=C(O)c1ccc(-c2ccc(C(=O)O)cc2)cc1]

As used herein, the term “substrate” refers to a reagent consumed during a catalytic reaction. The selective oxidation processes described herein typically include a first reaction zone wherein the substrate comprises one or more dimethylbiphenyl compounds. Optionally, the selective oxidation processes described herein may further include one or more additional reaction zones wherein the substrate typically comprises any unconsumed dimethylbiphenyl compound(s) and partially oxidized species contained in the reaction product from the preceding reaction zone.

As used herein, the term “semi-continuous reaction zone” refers to a reaction zone wherein the substrate is continuously supplied to the reaction zone for a period of time, e.g., to attain to a specified amount of the substrate, and wherein, during said continuous supply of the substrate, the reaction product of the reaction zone is not withdrawn from the reaction zone.

As used herein, the term “continuous reaction zone” refers to a reaction zone wherein the substrate is continuously supplied to the reaction zone and wherein the reaction product of the reaction zone is continuously withdrawn from the reaction zone.

Disclosed is a process for the selective oxidation of dimethylbiphenyl compounds, especially the 3,4′- and/or the 4,4′-isomers, to the corresponding biphenyldicarboxylic acids. In the process, at least one dimethylbiphenyl compound is supplied to at least one reaction zone together with an acidic solvent, an oxidizing medium, and a catalyst comprising cobalt, manganese, and bromine. The reaction zone is maintained under conditions including a temperature of 150 to 210° C. such that, in the presence of the Co/Mn/Br catalyst, the oxidizing medium selectively oxidizes the at least one dimethyl biphenyl compound to the fully oxidized dicarboxylic acid species. When a specified amount of the at least one dimethylbiphenyl compound has been supplied to the at least one reaction zone, for example after a given time of continuously supplying the at least one dimethylbiphenyl compound in a semi-continuous to reaction zone, further supply of the at least one dimethylbiphenyl compound is ceased while the supply of the oxidizing medium and catalyst is continued and the reaction zone is maintained under oxidation conditions including a temperature of 150 to 210° C. In this way, it is possible to recover from the reaction zone a product comprising at least 95 wt % of the biphenyl dicarboxylic acid based on the total weight of oxidized dimethylbiphenyl compound. In most cases, the reaction product comprises less than 1 wt % of the methylbiphenyl monocarboxylic acid and less than 2 wt % of the formylbiphenyl monocarboxylic acid, both based on the total weight of oxidized dimethylbiphenyl compound.

Any known process can be used to produce dimethylbiphenyl starting materials used in the present process, but preferably the process employs low cost feeds, such as toluene and/or benzene, as described in more detail below.

Production of Dimethyl-Substituted Biphenyl Compounds from Toluene

Often, the feed employed in the present processes comprises toluene, which is initially converted to (methylcyclohexyl)toluenes by reaction with hydrogen over a hydroalkylation catalyst according to the following reaction:

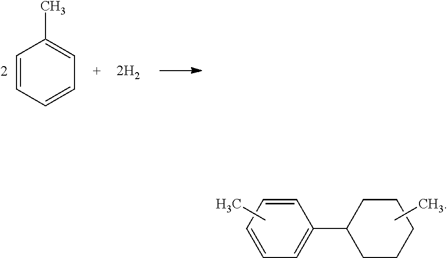

The catalyst employed in the hydroalkylation reaction is generally a bifunctional catalyst comprising a hydrogenation component and a solid acid alkylation component, typically a molecular sieve. The catalyst may also include a binder such as clay, alumina, silica and/or metal oxides. The latter may be either naturally occurring or in the form of gelatinous precipitates or gels including mixtures of silica and metal oxides. Naturally occurring clays which can be used as a binder include those of the montmorillonite and kaolin families, which families include the subbentonites and the kaolins commonly known as Dixie, McNamee, Georgia and Florida clays or others in which the main mineral constituent is halloysite, kaolinite, dickite, nacrite or anauxite. Such clays can be used in the raw state as originally mined or initially subjected to calcination, acid treatment or chemical modification. Suitable metal oxide binders include silica, alumina, zirconi a, titania, silica-alumina, silica-magnesia, silica-zirconia, silica-thoria, silica-beryllia, silica-titania as well as ternary compositions such as silica-alumina-thoria, silica-alumina-zirconia, silica-alumina-magnesia and silica-magnesia-zirconia.

Any known hydrogenation metal or compound thereof can be employed as the hydrogenation component of the catalyst, although suitable metals include palladium, ruthenium, nickel, zinc, tin, and cobalt, with palladium being particularly advantageous. The amount of hydrogenation metal present in the catalyst in any embodiment is between 0.05 and 10 wt %, such as between 0.1 and 5 wt % of the catalyst.

Often, the solid acid alkylation component comprises a large pore molecular sieve having a Constraint Index (as defined in U.S. Pat. No. 4,016,218) less than 2. Suitable large pore molecular sieves include zeolite beta, zeolite Y, Ultrastable Y (USY), Dealuminized Y (Deal Y), mordenite, ZSM-3, ZSM-4, ZSM-18, and ZSM-20. Zeolite ZSM-4 is described in U.S. Pat. No. 4,021,447. Zeolite ZSM-20 is described in U.S. Pat. No. 3,972,983. Zeolite Beta is described in U.S. Pat. No. 3,308,069, and Re. No. 28,341. Low sodium Ultrastable Y molecular sieve (USY) is described in U.S. Pat. Nos. 3,293,192 and 3,449,070. Dealwninized Y zeolite (Deal Y) may be prepared by the method found in U.S. Pat. No. 3,442,795. Zeolite UHP-Y is described in U.S. Pat. No. 4,401,556. Mordenite is a naturally occurring material but is also available in synthetic forms, such as TEA-mordenite (i.e., synthetic mordenite prepared from a reaction mixture comprising a tetraethylammonium directing agent). TEA-mordenite is disclosed in U.S. Pat. Nos. 3,766,093 and 3,894,104.

Alternatively, the solid acid alkylation component preferably comprises a molecular sieve of the MCM-22 family. The term “MCM-22 family material” (or “material of the MCM-22 family” or “molecular sieve of the MCM-22 family”), as used herein, includes one or more of: molecular sieves made from a common first degree crystalline building block unit cell, which unit cell has the MWW framework topology. (A unit cell is a spatial arrangement of atoms which if tiled in three-dimensional space describes the crystal structure. Such crystal structures are discussed in the “Atlas of Zeolite Framework Types”, Fifth edition, 2001, the entire content of which is incorporated as reference); molecular sieves made from a common second degree building block, being a 2-dimensional tiling of such MWW framework topology unit cells, forming a monolayer of one unit cell thickness, preferably one c-unit cell thickness; molecular sieves made from common second degree building blocks, being layers of one or more than one unit cell thickness, wherein the layer of more than one unit cell thickness is made from stacking, packing, or binding at least two monolayers of one unit cell thickness. The stacking of such second degree building blocks can be in a regular fashion, an irregular fashion, a random fashion, or any combination thereof; and molecular sieves made by any regular or random 2-dimensional or 3-dimensional combination of unit cells having the MWW framework topology.

Molecular sieves of MCM-22 family generally have an X-ray diffraction pattern to including d-spacing maxima at 12.4±0.25, 6.9±0.15, 3.57±0.07 and 3.42±0.07 Angstrom. The X-ray diffraction data used to characterize the material are obtained by standard techniques using the K-alpha doublet of copper as the incident radiation and a diffractometer equipped with a scintillation counter and associated computer as the collection system, Molecular sieves of MCM-22 family include MCM-22 (described in U.S. Pat. No. 4,954,325), PSH-3 (described in U.S. Pat. No. 4,439,409), SSZ-25 (described in U.S. Pat. No. 4,826,667), ERB-1 (described in European Patent No. 0293032), ITQ-1 (described in U.S. Pat. No. 6,077,498), ITQ-2 (described in International Patent Publication No. WO 97/17290), MCM-36 (described in U.S. Pat. No. 5,250,277), MCM-49 (described in U.S. Pat. No. 5,236,575), MCM-56 (described in U.S. Pat. No. 5,362,697) and mixtures thereof.

In addition to the toluene and hydrogen, the feed to the hydroalkylation reaction may include benzene and/or xylene which can undergo hydroalkylation to produce various methylated cyclohexylbenzene molecules of C₁₂ to C₁₆ carbon number. A diluent, which is substantially inert under hydroalkylation conditions, may also be included in the hydroalkylation feed, In certain embodiments, the diluent is a hydrocarbon, in which the desired cycloalkylaromatic product is soluble, such as a straight chain paraffinic hydrocarbon, a branched chain paraffinic hydrocarbon, and/or a cyclic paraffinic hydrocarbon. Examples of suitable diluents are decane and cyclohexane. Although the amount of diluent is not narrowly defined, desirably the diluent is added in an amount such that the weight ratio of the diluent to the aromatic compound is at least 1:100; for example at least 1:10, but no more than 10:1, desirably no more than 4:1.

The hydroalkylation reaction can be conducted in a wide range of reactor configurations including fixed bed, slurry reactors, and/or catalytic distillation towers. In addition, the hydroalkylation reaction can be conducted in a single reaction zone or in a plurality of reaction zones, in which at least the hydrogen is introduced to the reaction in stages. Suitable reaction temperatures are between 100° C. and 400° C., such as between 125° C. and 250° C., while suitable reaction pressures are between 100 and 7,000 kPa, such as between 500 and 5,000 kPa. The molar ratio of hydrogen to aromatic feed is typically from 0.15:1 to 15:1.

In the present process, it is found that MCM-22 family molecular sieves are particularly active and stable catalysts for the hydroalkylation of toluene or xylene. In addition, catalysts containing MCM-22 family molecular sieves exhibit improved selectivity to the 3,3′-dimethyl, the 3,4′-dimethyl, the 4,3′-dimethyl and the 4,4′-dimethyl isomers in the hydroalkylation product, while at the same time reducing the formation of fully saturated and heavy by-products. For example, using an MCM-22 family molecular sieve with a toluene feed, it is found that the hydroalkylation reaction product may comprise: at least 60 wt %, such as at least 70 wt %, for example at least 80 wt % of the 3,3′, 3,4′, 4,3′ and 4,4′-isomers of (methylcyclohexyl)toluene based on the total weight of all the (methylcyclohexyl)toluene isomers; less than 40 wt %, such as less than 30 wt %, for example from 15 to 25 wt % of the 2,2′, 2,3′, and 2,4′-isomers of (methylcyclohexyl)toluene based on the total weight of all the (methylcyclohexyl)toluene isomers; less than 30 wt % of methylcyclohexane and less than 2 wt % of dimethylbicyclohexane compounds; and less than 1 wt % of compounds containing in excess of 14 carbon atoms, such as di(methylcyclohexyl)toluene.

The hydroalkylation reaction product may also contain significant amounts of residual toluene, for example up to 50 wt %, such as up to 90 wt %, typically from 60 to 80 wt % of residual toluene based on the total weight of the hydroalkylation reaction product. The residual toluene can readily be removed from the reaction effluent by, for example, distillation. The residual toluene can then be recycled to the hydroalkylation reactor, together with some or all of any unreacted hydrogen. In some embodiments, it may be desirable to remove the C₁₄₊ reaction products, such as di(methylcyclohexyl)toluene, for example, by distillation.

The remainder of the hydroalkylation reaction effluent, composed mainly of (methylcyclohexyl)toluenes, is then dehydrogenated to convert the (methylcyclohexyl)toluenes to the corresponding methyl-substituted biphenyl compounds. The dehydrogenation is conveniently conducted at a temperature from 200° C. to 600° C. and a pressure from 100 kPa to 3550 kPa (atmospheric to 500 psig) in the presence of dehydrogenation catalyst. A suitable dehydrogenation catalyst comprises one or more elements or compounds thereof selected from Group 10 of the Periodic Table of Elements, for example platinum, on a support, such as silica, alumina or carbon nanotubes. In one embodiment, the Group 10 element is present in an amount from 0.1 to 5 wt % of the catalyst. In some cases, the dehydrogenation catalyst may also include tin or a tin compound to improve the selectivity to the desired methyl-substituted biphenyl product. In one embodiment, the tin is present in an amount from 0.05 to 2.5 wt % of the catalyst.

Particularly using an MCM-22 family-based catalyst for the upstream hydroalkylation reaction, the product of the dehydrogenation step comprises dimethylbiphenyl compounds in which the concentration of the 3,3′-, 3,4′- and 4,4′ isomers is at least 50 wt %, such as at least 60 wt %, for example at least 70 wt % based on the total weight of dimethylbiphenyl compounds. Typically, the concentration of the 2,X′-dimethylbiphenyl isomers in the dehydrogenation product is less than 50 wt %, such as less than 30 wt %, for example from 5 to 25 wt % based on the total weight of dimethylbiphenyl compounds.

Production of Dimethyl-Substituted Biphenyl Compounds from Benzene

In any embodiment the present processes for producing dimethyl-substituted biphenyl compounds employ benzene as the feed and comprises initially converting the benzene to biphenyl. For example, benzene can be converted directly to biphenyl by reaction with oxygen over an oxidative coupling catalyst as follows:

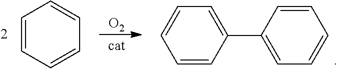

Details of the oxidative coupling of benzene can be found in Ukhopadhyay, Sudip; Rothenberg, Gadi; Gitis, Diana; Sasson, Yoel, 65(10) CASALI INSTITUTE OF APPLIED CHEMISTRY, HEBREW UNIVERSITY OF JERUSALEM, ISRAEL, JOURNAL OF ORGANIC CHEMISTRY 3107-3110 (2000), incorporated herein by reference.

Alternatively, benzene can be converted to biphenyl by hydroalkylation to cyclohexylbenzene according to the reaction:

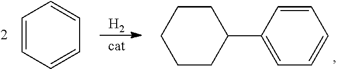

followed by dehydrogenation of the cyclohexylbenzene as follows:

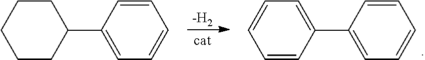

In such a process, the benzene hydroalkylation can be conducted in the same manner as described above for the hydroalkylation of toluene, while the dehydrogenation of the cyclohexylbenzene can be conducted in the same manner as described above for the dehydrogenation of (methylcyclohexyl)toluene.

Alternatively, benzene can be converted to biphenyl via thermal dehydro-condensation (i.e., contacting with heat), optionally conducted in the presence of steam. Direct dehydro-condensation of benzene to biphenyl is further described in Thompson, Q. E. 2000. Biphenyl and Terphenyls. Kirk-Othmer Encyclopedia of Chemical Technology.

In any case, the biphenyl product of the oxidative coupling step, dehydrocondensation, or the hydroalkylation/dehydrogenation sequence is then methylated, for example with methanol, to produce dimethylbiphenyl. Any known alkylation catalyst can be used for the methylation reaction, such as an intermediate pore molecular sieve having a Constraint Index (as defined in U.S. Pat. No. 4,016,218) of 3 to 12, for example ZSM-5.

The composition of the methylated product will depend on the catalyst and conditions employed in the methylation reaction, but inevitably will comprise a mixture of the different isomers of dimethylbiphenyl. Typically, the methylated product will contain from 50 to 100 wt % of 3,3′-, 3,4′- and 4,4′ dimethylbiphenyl isomers and from 0 to 50 wt % of 2,X′ (where X′ is 2′, 3′, or 4′)-dimethylbiphenyl isomers based on the total weight of dimethylbiphenyl compounds in the methylation product.

### Separation of Dimethylbiphenyl Isomers

Irrespective of the process used, the raw dimethylbiphenyl product from the production sequences described will contain unreacted components and by-products in addition to a mixture of dimethylbiphenyl isomers. For example, where the initial feed comprises toluene and the production sequence involves hydroalkylation to MCHT and dehydrogenation of the MCHT, the raw dimethylbiphenyl product will tend to contain residual toluene and MCHT and by-products including hydrogen, methylcyclohexane, dimethylcyclohexylbenzene, and C₁₄₊ heavy hydrocarbons in addition to the target dimethylbiphenyl isomers. Thus, often, prior to any separation of the dimethylbiphenyl isomers, the raw product of the MCHT dehydrogenation is subjected to one or more initial separation steps to remove at least part of the residues and by-products with significantly different boiling points from the desired dimethylbiphenyl isomers.

For example, the hydrogen by-product can be removed in a vapor/liquid separator and recycled to the hydroalkylation and/or MCHT dehydrogenation steps. The remaining liquid product can then be fed to one or more distillation columns to remove residual toluene and methylcyclohexane by-product, as well as effect initial separation of some of the lower boiling DMBP isomers. Thus, the normal boiling points and melting points of the dimethylbiphenyl isomers are shown in Table 1 below.

TABLE 1

| Isomer | Normal Boiling Point (° C.) | Melting Point (° C.) |
|---|---|---|
| 2,2′-Dimethylbiphenyl | 260.70 | — |
| 2,3′-Dimethylbiphenyl | 271.50 | — |
| 2,4′-Dimethylbiphenyl | 275.26 | −23.67 |
| 3,3′-Dimethylbiphenyl | 289.27 | 8.00 |
| 3,4′-Dimethylbiphenyl | 292.87 | 11.55 |
| 4,4′-Dimethylbiphenyl | 295.66 | 114.77 |

From Table 1 it will be seen that the similarity of the boiling points of the 3,3′, 3,4′ and 4,4′ DMBP isomers precludes their effective separation by distillation. However, the 2,X′ (where X′ is 2′, 3′, or 4′) isomers all have boiling points at least 15° C. below the 3,3′, 3,4′ and 4,4′ isomers and so can be readily separated from the latter by distillation. Thus, in any embodiment the liquid product of the MCHT dehydrogenation step is supplied to a distillation unit where the toluene is removed as overhead for recycle to the hydroalkylation unit, the unreacted MCHT and 2,X′-DMBP isomers are removed as an intermediate stream and the 3,3′, 3,4′ and 4,4′ DMBP isomers and heavy (C14+) by-products are separated as a bottoms stream.

This bottoms stream can then be supplied to a further distillation column to remove the 3,3′-, 3,4′- and 4,4′-DMBP isomers for recovery of at least the 3,4′ and 4,4′ DMBP isomers, while the heavies are conveniently purged from the system. Crystallization and other separation techniques can then, if desired, the recover the 3,4′ and 4,4′ DMBP isomers either as separate streams or as a mixed stream.

### Oxidation of Dimethylbiphenyl Isomer(s) to the Diacid

The DMBP isomer(s) recovered from the separation steps described above, normally comprising at least 3,4′- and/or 4,4′-DMBP, are then selectively oxidized to the corresponding, biphenyldicarboxylic acids. In any embodiment, the DMBP isomer(s) are supplied to at least one reaction zone together with an acidic solvent, an oxidizing medium, and a catalyst comprising cobalt, manganese, and bromine. Suitable acidic solvents include acetates, carbonates, acetate tetrahydrates, and bromides. In some cases, the acidic solvent comprises a C₂ to C₄ monocarboxylic acid, especially acetic acid. The DMBP isomer(s) can be supplied to the at least one reaction zone as a solution in the acidic solvent, such as a solution comprising from 1 to 20 wt % of at least one dimethylbiphenyl compound, or the DMBP isomer(s) and acidic solvent can be supplied separately to the reaction zone. Any oxidant, such as oxygen, ozone or air, or any other oxygen source, such as hydrogen peroxide, can be used as the oxidizing medium but in most cases the oxidizing medium comprises air.

The catalyst comprises cobalt, manganese_{;} and bromine, typically such that the atomic ratio of manganese to cobalt in the catalyst is from 0.01 to 3.0 and the atomic ratio of bromine to the combined amount of manganese and cobalt in the catalyst is from 0.01 to 1.5. Any bromine compound such as HBr, Br₂, tetrabromoethane, and benzyl bromide may be used as a source of bromine. Suitable sources of manganese and cobalt include any compound soluble in acidic solvent (e.g., acetates, carbonates, acetate tetrahydrates and bromides).

Advantageously, the supply of the at least dimethylbiphenyl compound may be carried out in a semi-continuous or continuous reaction zone, particularly where the substrate to comprises 4,4′-DMBP. Without wishing to be bound by theory, it is believed that supplying the at least one dimethylbiphenyl compound in a semi-continuous or continuous reaction zone alters the oxidation reaction pathways as compared to a batch reaction zone, particularly in the case where the substrate comprises 4,4′-DMBP, resulting in reduced formation of undesired under-oxidized species as well as a reduction in catalyst deactivation. In any embodiment the at least dimethylbiphenyl compound is supplied in a semi-continuous or continuous reaction zone, the oxidizing medium is typically continuously added and withdrawn from the reaction zone.

The reaction zone is maintained under conditions including a temperature of 150 to 210° C., preferably 160 to 200° C., such that, in the presence of the Co/Mn/Br catalyst, the oxidizing medium oxidizes the at least one dimethylbiphenyl compound towards the fully oxidized dicarboxylic acid species. When a specified amount of the at least one dimethylbiphenyl compound has been supplied to the at least one reaction zone, for example after a given time of continuously supplying the at least one dimethylbiphenyl compound in a semi-continuous operation, further supply of the at least one dimethylbiphenyl compound is ceased while the supply of the oxidizing medium and catalyst is continued and the reaction zone is maintained under oxidation conditions including a temperature of 150 to 210° C.

In any embodiment the at least one dimethylbiphenyl compound is supplied to a continuous reaction zone, the at least one dimethylbiphenyl compound, the acidic solvent, the oxidizing medium, and the catalyst are supplied to a first continuous reaction zone in a first oxidation step and the oxidizing medium and catalyst (without the dimethylbiphenyl compound) are supplied in a second oxidation step to a second continuous reaction zone, which typically receives the entire effluent from the first reaction zone. In any embodiment the mean residence time of the second reaction zone typically ranges from between 10% to 400% of the mean residence time of the first reaction zone.

Using the oxidation process described above, it is possible to recover from the at least one reaction zone a product comprising at least 95 wt % of the biphenyldicarboxylic acid derivative(s) of the starting dimethylbiphenyl isomer(s) based on the total weight of oxidized dimethylbiphenyl compound. In most cases, the reaction product comprises less than 1 wt %, preferably less than 0.1 wt %, of the methylbiphenyl monocarboxylic acid and less than 2 wt %, preferably less than 0.1 wt %, of the formylbiphenyl monocarboxylic acid, both based on the total weight of oxidized dimethylbiphenyl compound.

The recovered biphenyldicarboxylic acid can then be fed directly to polymerization or esterified to form a diester for subsequent polymerization.

Particularly preferably, polyesters may be prepared from the diacid or diester, ether by conventional direct esterification or transesterification methods. Suitable diols for reaction with the above-mentioned diester or diacid compositions include alkanediols having 2 to 12 carbon atoms, such as monoethylene glycol, diethylene glycol, 1,3-propanediol, or 1,4-butane diol, 1,6-hexanediol, and 1,4-cyclohexanedimethanol. Optionally, the diester or diacid compositions may be further reacted with terephthalic acid or terephthalate. Suitable catalysts include but not limited to titanium alkoxides such as titanium tetraisopropoxide, dialkyl tin oxides, antimony trioxide, manganese (II) acetate and Lewis acids. Suitable conditions include a temperature 170 to 350° C. for a time from 0.5 hours to 10 hours. Generally, the reaction is conducted in the molten state and so the temperature is selected to be above the melting point of the monomer mixture but below the decomposition temperature of the polymer. A higher reaction temperature is therefore needed for higher percentages of biphenyl dicarboxlic acid in the monomer mixture. The polyester may be first prepared in the molten state followed by a solid state polymerization to increase its molecular weight or intrinsic viscosity for applications like bottles.

The invention will now be more particularly described with reference to the following non-limiting Examples.

### HPLC Method

Product and impurity concentrations of the solid and mother liquor fractions recovered from the selective oxidation reactions in the following examples were determined using high performance liquid chromatography (HPLC), The samples were analyzed on an Agilent Technologies 1100 Series system equipped with a Phenomenex™ Synergi Hydro-RP phase column (100×2 mm inner diameter and 2.5 μm particles) and DAD detector (254 and 280 nm). The HPLC was performed at 23° C. with an eluent rate of 0.4 ml/min. The composition of the mobile phase was 80/20 water (0.1% TFA)/ACN for the initial 10 minutes, and subsequently linearly ramped to 35/65 water (0.1% TFA)/ACN over a period of 40 minutes. The response factor of the different components were determined using 40 ppm dilutions in DMSO.

### EXAMPLE 1

### Semi-Continuous Oxidation of 3,4′-DMBP with no Post-Oxidation

A semi-continuous oxidation test was conducted where 3,4′-DMBP was fed along with a constant flow of air into a solution of Co/Mn/Br catalyst and solvent pre-equilibrated at the reaction temperature. The test was conducted at a reaction temperature 190° C. and 200 psig (1400 kPa-g) with an air feed rate set at 752 sccm, using a catalyst comprising Co/Mn/Br concentrations of 800/800/1200 ppmw, and 95 wt % acetic acid/5 wt % water as the solvent. The 3,4′-DMBP addition was continued for 2 hours at a rate sufficient to achieve a final concentration of 15 wt % DMBP in solution in the absence of oxidation (i.e., 0.253 ml/min based on the density of 3,4′-DMBP at 25° C., 0.9932 g/ml), after which the supply of 3,4′-DMBP was terminated, the flowing air was switched to nitrogen (i.e., there was no post-oxidation time), and the external heat source was switched off to reduce the temperature to is ambient temperature. A crude filtration was performed to separate the solids from the mother liquor using a small amount of 95 wt % acetic acid/5 wt % water solvent to rinse the solids. The solids were dissolved and diluted in dimethylacetamide and analyzed by HPLC to quantify the amount of impurities. The measured amount of impurities was then subtracted from the total weight of the solids to determine the product yield in the solids. Separately, the mother liquor was diluted in dimethylacetamide and analyzed by HPLC to quantify the dissolved amount of desired product and impurities. The results of these analyses are shown in Table 2, including the % solids recovery and its purity. The selectivities and yields shown in Table 2 are based on the total amounts recovered in both the solids and mother liquor.

### EXAMPLE 2

### Semi-Continuous Oxidation of 4,4′-DMBP with No Post-Oxidation

The procedure conducted in Example 1 was repeated but using 4,4′-DMBP as the dimethylbiphenyl isomer, with the results also shown in Table 2.

### EXAMPLE 3

### Semi-Continuous Oxidation of 4,4′-DMBP with Post-Oxidation

The procedure conducted in Example 2 was repeated, but instead of switching the flowing air to nitrogen immediately after terminating the supply of 4,4′-DMBP, the supply of air was continued for an additional 1 hour (i.e., post-oxidation time). The results of Example 3 are again shown in Table 2. As seen from a comparison of Examples 2 and 3 in Table 2, the inclusion of post-oxidation time resulted in improved selectivity of the oxidation. For instance, Example 3 (including post-oxidation time) demonstrated a decrease in undesired aldehyde-acid formation from a molar yield of 2.2% to 1.1% in comparison with Example 2 (not including post-oxidation time).

TABLE 2
Semi-Continuous Reaction Data

| Example | 1 | 2 | 3 |
|---|---|---|---|
| Substrate | 3,4′- | 4,4′-DMBP | 4,4′-DMBP |
| | DMBP | | |
| Post-Oxidation Tune (h) | 0 | 0 | 1 |
| DMBP Balance (%) | 93.2% | 91.6% | 95.0% |
| Conversion (%) | 82.8% | 99.8% | 99.9% |
| Yield of solids (%) | 26.8% | 90.2% | 93.8% |
| Product purity in solids (%) | 98.1% | 97.1% | 98.9% |
| % Acid-Aid in solids (%) | 0.6% | 2.6% | 1.1% |
| Total Yield of Desired Product (%) | 28.7% | 88.0% | 93.1% |

Mol % Selectivity

| | | | |
|---|---|---|---|
| Diacid | 79.7% | 96.3% | 98.2% |
| Ald-Acid | 1.2% | 2.6% | 1.1% |
| M-Acid | 5.2% | 0.2% | 0.0% |
| M-Ald | 8.1% | 0.0% | 0.0% |
| Total Identified Underreacted | 42.2% | 3.6% | 1.4% |
| Other Identified | 0.8% | 0.4% | 0.5% |

Mol % Yield

| | | | |
|---|---|---|---|
| Diacid | 60.4% | 88.0% | 93.1% |
| Ald-Acid | 0.9% | 2.4% | 1.1% |
| M-Acid | 3.9% | 0.2% | 0.0% |
| M-Ald | 6.1% | 0.0% | 0.0% |
| Total Identified Underreacted | 14.8% | 3.3% | 1.4% |
| Other Identified | 0.6% | 0.3% | 0.5% |
| Reactor % Mass Balance | 96.1% | 90.9% | 93.6% |

### EXAMPLE 4

### Batch Oxidation of 3,4′-DMBP

A batch oxidation test was conducted where 3,4′-DMBP was mixed in a solution of Co/Mn/Br catalyst and solvent at 23° C. in an amount sufficient to achieve a concentration of 10 wt % 3,4′-DMBP. The solution was rigorously stirred, purged with nitrogen, and pre-equilibrated at reaction temperature before beginning the flow of air. The test was conducted at 170° C. and 500 psig (3450 kPa-g) with an air feed rate set at 1500 sccm, using a catalyst comprising Co/Mn/Br concentrations of 800/800/1200 ppmw, and 95 wt % acetic acid/5 wt % water as the solvent. The air feed rate was set to 1500 sccm. The reaction proceeded for 1 hour after which the flowing air was switched to nitrogen, and the external heat source was switched off to reduce the temperature to ambient temperature. Separation and analysis of the solids and mother liquor was then performed using the procedure described in Example 1. The results of the analyses are shown in Table 3, including the % solids recovery and its purity. The selectivities and yields shown in Table 3 are based on the total amounts recovered in both the solids and mother liquor.

### EXAMPLE 5

### Batch Oxidation of 4,4′-DMBP

The process of Example 4 was repeated but using 4,4′-DMBP as the dimethylbiphenyl isomer, with the results also shown in Table 3.

TABLE 3
Batch Reaction Data

| Example | 4 | 5 |
|---|---|---|
| Substrate | 3,4′-DMBP | 4,4′-DMBP |
| DMBP Balance (%) | 94.0% | 90.7% |
| Conversion (%) | 100% | 100% |
| Yield of solids (%) | 87.5% | 89.0% |
| Product purity in solids (%) | 96.9% | 82.5% |
| % Acid-Ald in solids (%) | 2.8% | 13.2% |
| Total Yield of Desired Product (%) | 86.2% | 73.7% |

Mol % Selectivity

| | | |
|---|---|---|
| Diacid | 94.9% | 81.9% |
| Ald-Acid | 3.1% | 13.1% |
| M-Acid | 0.1% | 2.8% |
| M-Ald | 0.0% | 0.0% |
| Total Identified Underreacted | 4.1% | 16.8% |
| Other Identified | 1.0% | 1.3% |

Mol % Yield

| | | |
|---|---|---|
| Diacid | 86.2% | 73.7% |
| Ald-Acid | 2.8% | 11.8% |
| M-Acid | 0.1% | 2.6% |
| M-Ald | 0.0% | 0.0% |
| Total Identified Underreacted | 3.7% | 15.1% |
| Other Identified | 0.9% | 1.2% |
| Reactor % Mass Balance | 96.6% | 95.8% |

As can be seen from a comparison of Tables 2 and 3, batch oxidation of 4,4′-DMBP resulted in significantly higher production of identified underreacted species as compared to semi-continuous oxidation (15.1% in Example 5 as compared to 3.6% in Example 2 and 1.3% in Example 3).

While the present invention has been described and illustrated by reference to particular embodiments, those of ordinary skill in the art will appreciate that the invention lends itself to variations not necessarily illustrated herein. For this reason, then, reference should be made solely to the appended claims for purposes of determining the true scope of the present invention. All documents described herein are incorporated by reference herein, including any priority documents and/or testing procedures to the extent they are not inconsistent with this text. Likewise, the term “comprising” is considered synonymous with the term “including,” and whenever a composition, an element or a group of elements is preceded with the transitional phrase “comprising”, it is understood that we also contemplate the same composition or group of elements with transitional phrases “consisting essentially of,” “consisting of”, “selected from the group of consisting of,” or “is” preceding the recitation of the composition, element, or elements and vice versa.

1. A process for selective oxidation of at least one dimethylbiphenyl compound to the corresponding biphenyldicarboxylic acid, the process comprising:
(a1) supplying at least one dimethylbiphenyl compound, an acidic solvent, an oxidizing medium, and a catalyst comprising cobalt, manganese, and bromine to at least one reaction zone;
(b1) contacting the at least one dimethylbiphenyl compound and the oxidizing medium with the catalyst in the at least one reaction zone at a temperature of 150 to 210° C. to oxidize the at least one dimethylbiphenyl compound;
(c1) ceasing the supply of the at least one dimethylbiphenyl compound but continuing the supply of the oxidizing medium and catalyst to the at least one reaction zone at a temperature of 150 to 210° C.; and
(d1) recovering from the at least one reaction zone a reaction product comprising at least 95 wt % of the corresponding biphenyldicarboxylic acid based on the total weight of oxidized dimethylbiphenyl compound.

2. The process of claim 1, wherein the biphenyldicarboxylic acid is 3,4′-biphenyldicarboxylic acid, 4,4′-biphenyldicarboxylic acid, or a mixture thereof.

3. The process of claim 1, wherein the acidic solvent comprises a C2 to C4 monocarboxylic acid.

4. The process of claim 3, wherein the C2 to C4 monocarboxylic acid comprises acetic acid.

5. The process of claim 1, wherein the supplying of step (a1) is carried out in at least one semi-continuous reaction zone.

6. The process of claim 1, wherein the supplying of step (a1) is carried out in at least one continuous reaction zone.

7. The process of claim 6, wherein the at least one dimethylbiphenyl compound, the acidic solvent, the oxidizing medium and the catalyst are supplied to a first continuous reaction zone in (a1) and the oxidizing medium and catalyst are supplied to a second continuous reaction zone in (c1), wherein the second continuous reaction zone receives at least part of the effluent from the first continuous reaction zone.

8. The process of claim 1, wherein the at least one dimethylbiphenyl compound is supplied to the at least one reaction zone as a solution in the acidic solvent.

9. The process of claim 8, wherein the solution comprises from 1 to 20 wt % of the at least one dimethylbiphenyl compound.

10. The process of claim 1, wherein the oxidizing medium comprises oxygen.

11. The process of claim 1, wherein the atomic ratio of manganese to cobalt in the catalyst is from 0.01 to 3.0.

12. The process of claim 1, wherein the atomic ratio of bromine to the combined amount of manganese and cobalt in the catalyst is from 0.01 to 1.5.

13. The process of claim 1, wherein the reaction product from the at least one reaction zone comprises less than 1 wt % of the methylbiphenyl monocarboxylic acid based on the total weight of oxidized dimethylbiphenyl compound.

14. The process of claim 1, wherein the reaction product from the at least one reaction zone comprises less than 2 wt % of the formylbiphenyl monocarboxylic acid based on the total weight of oxidized dimethylbiphenyl compound.

15. A polyester product produced from the reaction of a diol with at least one of the following;
(i) the biphenyldicarboxylic acid recovered in step (d1) according to any one of claims 1 to 14; and/or
(ii) a biphenyldiester product formed from the esterification of the biphenyldicarboxylic acid recovered in step (d1) according to claim 1.

16. A process for producing 3,4′- and/or 4,4′-biphenyldicarboxylic acid, the process comprising:
(a2) supplying 3,4′- and/or 4,4′-dimethylbiphenyl, an acidic solvent, an oxidizing medium, and a catalyst comprising cobalt, manganese, and bromine to at least one reaction zone;
(b2) contacting the 3,4′- and/or 4,4′-dimethylbiphenyl and the oxidizing medium with the catalyst in the at least one reaction zone at a temperature of 150 to 210° C. to oxidize the at least one dimethylbiphenyl compound;
(c2) ceasing the supply of the 3,4′- and/or 4,4′-dimethylbiphenyl but continuing the supply of the oxidizing medium and catalyst to the at least one reaction zone at a temperature of 150 to 210° C.; and
(d2) recovering from the at least one reaction zone a reaction product comprising at least 95 wt % of 3,4′- and/or 4,4′-biphenyldicarboxylic acid based on the total weight of oxidized 3,4′- and/or 4,4′-dimethylbiphenyl.

17. The process of claim 16, wherein the acidic solvent comprises a C₂ to C₄ monocarboxylic acid.

18. The process of claim 17, wherein the C₂ to C₄ monocarboxylic acid comprises acetic acid.

19. The process of claim 16, wherein the supplying of step (a2) is carried out in at least one semi-continuous reaction zone.

20. The process of claim 16, wherein the supplying of step (a2) is carried out in at least one continuous reaction zone.

21. The process of claim 20, wherein the 3,4′- and/or 4,4′-dimethylbiphenyl, the acidic solvent, the oxidizing medium and the catalyst are supplied to a first continuous reaction zone in (a2) and the oxidizing medium and catalyst are supplied to a second continuous reaction zone in (c2), wherein the second continuous reaction zone receives at least part of the effluent from the first continuous reaction zone.

22. The process of claim 16, wherein the oxidizing medium comprises oxygen.

23. The process of claim 16, wherein the atomic ratio of manganese to cobalt in the catalyst is from 0.01 to 3.0.

24. The process of claim 16, wherein the reaction product from the at least one reaction zone comprises less than 1 wt % of the methylbiphenyl monocarboxylic acid based on the total weight of oxidized 3,4′- and/or 4,4′-dimethylbiphenyl.

25. The process of claim 16, wherein the reaction product from the at least one reaction zone comprises less than 2 wt % of the formylbiphenyl monocarboxylic acid based on the total weight of oxidized 3,4′- and/or 4,4′-dimethylbiphenyl.
